(19) United States
(12) Patent Application Publication
(10) Pub. No.: US 2006/0040980 A1
(43) Pub. Date: Feb. 23, 2006

(54) Ionophores as cancer chemotherapeutic agents

(76) Inventors: Stuart E. Lind, Oklahoma City, OK (US); Wei-Qun Ding, Edmonds, OK (US)

Correspondence Address: REED SMITH LLP, 1301 K STREET, N.W., SUITE 1100 EAST TOWER, WASHINGTON, DC, 20005, US

(21) Appl. No.: 11/206,818

(22) Filed: Aug. 19, 2005

Related U.S. Application Data
(60) Provisional application No. 60/603,352, filed on Aug. 20, 2004.

(51) Int. Cl. A61K 31/47; A61K 31/44; A61K 31/4412

(52) U.S. Cl. 514/310; 514/312; 514/345

(57) ABSTRACT

This invention relates to anti-cancer uses of ionophores of which clioquinol (5-chloro-7-iodo-8-hydroxyquinoline) is a prototype drug. The present invention is further directed toward using ionophores such as clioquinol alone, or in combination with metals (e.g., zinc or copper, manganese) as anti-cancer and anti-angiogenic agents. This invention further relates to the potentiation of the anti-cancer properties of polyunsaturated fatty acids when used in conjunction with the ionophores of the present invention. The invention is also directed to the therapeutic or prophylactic use of pharmaceutical compositions containing the ionophores of the present invention, and to methods of treating cancer as well as other disease states associated with unwanted angiogenesis and/or cellular proliferation, such as diabetic retinopathy, neovascular glaucoma, rheumatoid arthritis, and psoriasis, by administering effective amounts of such compounds.

This Application claims priority to U.S. Provisional Application No. 60/603,352 filed Aug. 20, 2004.

### FIELD OF THE INVENTION

This invention relates to the use of anti-microbial metal chelators as anti-tumor agents. In particular, the present invention relates to anti-cancer uses of ionophores of which clioquinol (5-chloro-7-iodo-8-hydroxyquinoline) is a prototype drug. The present invention in various forms is directed toward using ionophores such as clioquinol alone, or in combination with metal (e.g., zinc or copper, manganese) as anti-cancer and anti-angiogenic agents. This invention further relates to the potentiation of the anti-cancer properties of polyunsaturated fatty acids when used in conjunction with the ionophores of the present invention.

### BACKGROUND OF THE INVENTION

It has long been known that metals such as copper and iron are involved in carcinogenesis (1-3) and angiogenesis (4). Metal chelators such as desferrioxamine, an iron chelator (5), and tetrathiomolybdate, a copper chelator, (6) have been shown to suppress tumor growth, tumor metastases and angiogenesis, and are currently being investigated in clinical trials (5, 7, 8). Metal chelators may exert anti-tumor effects in a variety of ways. Iron chelators have been shown to induce apoptosis through a p53-independent pathway (9) as well as by the inhibition of N-myc expression (10), while copper chelators induce apoptosis of tumor cells through the inhibition of the NF kappa B signaling cascade (11).

Clioquinol (5-chloro-7-iodo-8-hydroxyquinoline) is a chelator of copper, iron and zinc. It was first prepared in Germany in the early part of the last century (12) and was used for many years as an anti-microbial for the treatment of a variety of infectious diseases, notably diarrhea and skin infection. In the late 1960s it was linked to the appearance in Japan of an epidemic of a rare neurological syndrome, subacute myelooptic neuropathy (SMON). However, subsequent epidemiological analysis has brought the link between SMON and clioquinol into question and it has been used safely in recent clinical trials for human subjects with Alzheimer's disease.

Clioquinol's introduction into modem human studies was prompted by work in a mouse model of Alzheimer's disease which was based upon the hypothesis that clioquinol's metal-chelating properties would prove beneficial in the treatment of Alzheimer's disease (14). Clioquinol has been recently found to be useful in animal models of Alzheimer's and Parkinson's diseases, and has been used without toxic neurological effects in two recent clinical trials.

Hyperproliferative disease states, including cancer, are characterized by cells rampantly winding through the cell cycle with uncontrolled vigor due to, for example, damage to the genes that directly or indirectly regulate progression through the cycle. Thus, agents that modulate the cell cycle, and hyperproliferation, could be used to treat various disease states associated with uncontrolled or unwanted cell proliferation. Moreover, the applicability of antiproliferative agents may be expanded to treating cardiovascular maladies such as artherosclerosis or restenosis (See Braun-Dullaeus et al., Circulation, 98, 82-89 (1998)), and states of inflammation, such as arthritis (See, Taniguchi et al., Nature Med., 5, 760-767(1999)) or psoriasis.

Mechanisms of cell proliferation are under active investigation at cellular and molecular levels. At the cellular level, de-regulation of signaling pathways, loss of cell cycle controls, unbridled angiogenesis or stimulation of inflammatory pathways are under scrutiny, while at the molecular level, these processes are modulated by various proteins, among which protein kinases are prominent suspects. Overall abatement of proliferation may also result from programmed cell death, or apoptosis, which is also regulated via multiple pathways, some involving proteolytic enzyme proteins.

### BRIEF SUMMARY OF THE INVENTION

It is an object of the present invention to provide a method of treating cancer and or inhibiting angiogenesis using ionophores, preferably, clioquinol. The present inventors have found that clioquinol kills human cancer cells by apoptosis, with IC₅₀s in the low micromolar range. Pharmacokinetic studies performed in one of the recent human clinical trials revealed that oral administration of clioquinol results of in trough blood levels of about 25 μM, levels that are equivalent to the IC₅₀s in testing clioquinol in vitro against a variety of human cancer cell lines. The instant inventors have demonstrated that clioquinol does not work simply by chelating metals, but by acting as an ionophore and its anti-cancer effects can be potentiated by zinc, copper, and iron. Although copper and iron are well recognized to be cytotoxic by virtue of increasing the formation of hydroxyl radicals, zinc, which is not redox active, has not generally been considered a cytotoxic metal. Because zinc and copper are known to bind to albumin, and albumin is taken up preferentially by tumors, albumin can be utilized to administer both clioquinol and one or more of these metals.

It is therefore another object of the present invention to provide a method for treating cancer and/or inhibiting angiogenesis using ionophores such as clioquinol as a single agent; ionophores such as clioquinol administered simultaneously with zinc, copper, iron or manganese; ionophores such as clioquinol administered with albumin-metal complexes, and said ionophores may be administered at various times prior to or preferably after administering the metal or the albumin-metal complexes. Such compounds may be administered by a variety of routes to treat or prevent cancer or conditions wherein blood vessel growth is detrimental to the host such as in blood vessel tumors, cancer of any etiology, hyper proliferation of blood vessels in hypoxic or hyperglycemic tissues such as in the eyes of diabetic patients.

It is thus a further object of the invention to provide clioquinol (alone or with other compounds or metals shown herein) to be administered to humans and/or other mammals for treatment of various cancer (e.g., those indicated in Table 1) and blood vessel proliferative diseases using protocols as described in U.S. Pat. No. 6,323,218 (Col. 15-18) and U.S. Pat. 6,001,852 (Col. 6-10), for example, each of which is hereby expressly incorporated herein by reference in its entirety. Other derivatives of clioquinol such as 8-hydroxyquinoline (8-HQ), and 5,7 di-iodo-8-HQ, chloroxine (5, 7-dichloro -8-hydroxyquinolone), oxine (8-hydroxyquinolone), as well as other well known clioquinol derivatives can be used interchangeably or in combination with clioquinol in accordance with the present invention. Other ionophores and/or other agents including pyrithione and dithiocarbamates such as carbamodithioic acid, pyrrolidine dithiocarbamate, prolinedithiocarbamate, disulfiram, diethyldithiocarbamate, pyrrolidine dithiocarbamate, dimethyldithiocarbamate, diethylthiocarbamate, diethyldithiocarbamate ion, dimethylthiocarbamate, and dibenzyldithiocarbamate are also contemplated as anti-cancer agents to be used herein, alone or in combination with other agents described herein. The clioquinol or other agents may be used with metals, including copper, zinc and iron, and may be supplied alone or in combination with the anti-cancer agent, or may be supplied with a carrier such as albumin. The clioquinol and/or other anti-cancer agent or agents may be administered before, with, or after administration of the metal or metal complex when a metal or metal complex is used in the treatment.

Additionally, the inventors have been studying the anti-cancer properties of docosahexaenoic acid (DEA), an n-3 polyunsaturated fatty acid that kills cancer cells as a result of undergoing lipid peroxidation (15). Since clioquinol is a copper and zinc chelator, the inventors sought to use it to inactivate a key intracellular anti-oxidant enzyme, superoxide dismutase-1 (SOD 1), which requires zinc and copper to function) and potentiate DHA's cytotoxicity. Using isobolographic analysis, the inventors found that clioquinol did indeed potentiate DHA, but also exhibited significant cytotoxic effects by itself (Table 1).

It is yet another object of the invention to provide a method of using ionophores such as clioquinol alone, or in combination with metals or albumin-metal complexes and further in combination with polyunsaturated fatty acids including but not limited to docosahexaenoic acid, conjugated docosahexaenoic acid, eicosapentaenoic acid, conjugated eicosapentaenoic acid, alpha or beta—eleostearic acid, punicic acid, parinaric acid, linoleic acid isomers; to act as tumor-selective cytotoxic agents.

TABLE 1
IC₅₀ (μM) of clioquinol towards human tumor cell lines (n = 3)

| Cell line | IC₅₀ (μM) |
|---|---|
| DHL-4 (B-cell) | 6.7 |
| Raji (B-cell) | 12.4 |
| A2780 (ovarian) | 14.2 |
| MDA-MB-231 (breast) | 19.9 |
| T24 (Bladder) | 20 |
| M-panc-96 (pancreas) | 23.6 |
| MCF-7 (breast) | 29.7 |
| SiHa (cervical) | 38.8 |

Inventors had thought that clioquinol was active against tumor cells because it was a metal chelator, but found that simple chelation could not explain all of the results. Most importantly, experiments in which various metals were added to clioquinol-treated cells revealed that these metals (copper, zinc and iron) potentiated clioquinol's activity, suggesting that clioquinol was acting as an ionophore, a class of compounds known to chemists and biologists, but not commonly recognized to have anti-cancer properties. lonophores such as monensin, lasalocid, laidlomycin, salinomycin and narasin have anti bacterial properties, and are used to increase feed efficiency of ruminant animals (16), but are not currently used therapeutically in humans. It appears that clioquinol has its anti-cancer effect due to its ability to act as an ionophore. Further, ionophores, a novel class of anti-cancer agents, can be administered with key metals (such as copper and zinc) to selectively kill cancer cells.

Thus a further aspect of this invention is the use of pharmaceutical compositions comprising ionophores, either alone or in combination with key metals such as copper, zinc, iron, manganese, as an anti-cancer and anti-angiogenic therapeutic, said pharmaceutical compositions optionally further comprising albumin and or polyunsaturated fatty acids.

### BRIEF DESCRIPTION OF THE DRAWINGS

FIG. 1 shows clioquinol potentiated killing of tumor cells by docosahexaneoic acid. Raji cells (a human B-cell Burkitt lymphoma line) were treated with clioquinol (A), DHA (B) or clioquinol plus DHA (C). Cell viability was determined with the MTS assay. IC₅₀ was calculated using nonlinear regression analysis (Prism 3.0). The concentration of clioquinol and DHA used in (C) was based on the ratio of IC₅₀s for clioquinol and DHA (12.4 μM and 85 μM, respectively). Shown in D is the isobologram analysis indicating a synergy of clioquinol and DHA in killing Raji cells. Data (n=3) are presented as means±S.E.M.

FIG. 2 shows X-band BSR spectra of Raji cells revealing loss of tyrosyl signal from ribonucleotide reductase by clioquinol treatment. Cell lysates were prepared from Raji cells incubated in media for 6 hours alone (lower trace) or with 10 μM clioquinol (CQ, upper trace). Background signals were subtracted to enhance the tyrosyl radical signal. Spectrometer conditions: microwave freq. 9.632 GHz, microwave power 1 mW, time constant 0.16384 sec., mod. freq. 100 KHz, mod. amplitude 10 G, temp. 30 K, 10 scans averaged.

FIG. 3 shows the potentiation of clioquinol-induced cytotoxicity towards Raji cells by metals.

FIG. 4 demonstrates that clioquinol (CQ) is a zinc ionophore. A2789 cells were treated with increasing concentrations of zinc in the presence of clioquinol or the zinc ionophore pyrithione and the fluorescent zinc indicator FluoZin-3.

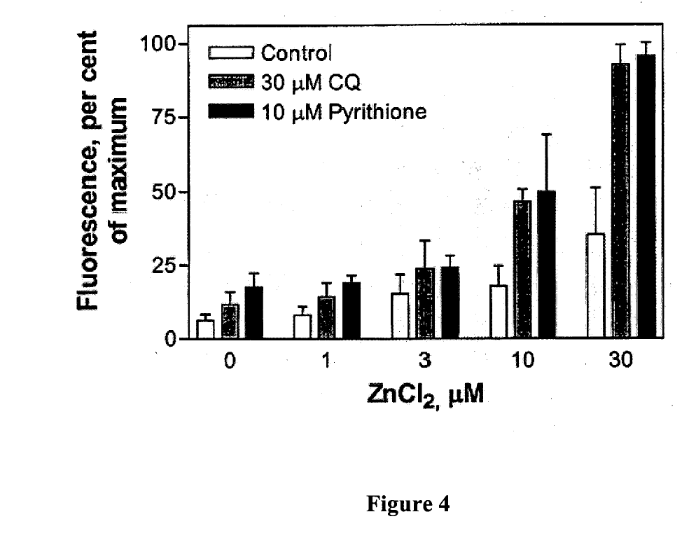

FIG. 5 shows clioquinol induced apoptosis in Raji cells through caspase dependent mechanism.

FIG. 6 shows the effects of clioquinol on growth of a human E-cell lymphoma line in nude mice.

FIG. 7 shows the effects of clioquinol on growth of a human ovarian cancer cell line in nude mice.

FIG. 8 shows a comparative study of endothelial cell tube formation between cells treated with bFGF and without bFGF plated on Matrigel after 21 hours of incubation.

FIG. 9 shows endothelial cell tube formation inhibition at different concentrations of clioquinol.

FIG. 10 shows a correlation between cell survival and tube branches and the anti-angiogenic effect of clioquinol.

FIG. 11 shows a correlation between cell survival and tube branches and the anti-angiogenic effect of pyrithione.

FIG. 12 shows a correlation between cell survival and tube branches and the anti-angiogenic effect of pyrithione.

### DETAILED DESCRIPTION OF THE PREFERRED EMBODIMENTS OF THE INVENTION

Materials

The CellTiter 96® Aqueous ONE Solution (Promega) was used to assess cellular viability via reduction of (3-(4,5-dimethylthiazol-2-yl)-5-(3carboxymethoxyphenyl)-2-(4-sulfophenyl)-2H-tetrazolium (MTS). An ELISA-based apoptosis assay kit was from Roche, and an anti-poly (ADP-ribose) polymerase (PARP) monoclonal antibody was from BIOMOL Research Laboratories Inc. Nude mice were from Animal Technology Limited. All other reagents were analytic grade.

Cell Culture and Viability Assay

DHL-4 cells were provided by Dr. Linda Boxer (Stanford University), A2780 cells by Dr. Stephen Howell (University of California, San Diego), SiHa cells by Dr. Doris Benbrook (University of Oklahoma). All other cells were obtained from the ATCC. Cells were routinely grown in a 75-mm flask, in an environment containing 5% CO2, and passed every three days. Cell viability was analyzed using the MTS assay. Depending on growth properties of each cell line, 2,000-20,000 cells were plated in each well of a 96-well tissue culture plate with 100 μL of medium, which resulted in 40-50% confluence of the cells after 24 h of growth. The medium was then replaced with 100 μL of fresh medium containing clioquinol or other reagents, and the cells were grown for designated periods of time. The MTS assay was performed using the protocol provided by the manufacturer. In short, 20 μL of MTS solution was added to each well, and cells were incubated at 37° C. for 1-2 h. The optical density (490 nm) of each well was then determined. Data are presented as a percentage of the values obtained from cells cultured under the same conditions in the absence of clioquinol or other chemicals.

Western Blot Analysis

Western blot analysis was performed as previously described (57-59). Briefly, cells were lysed in a lysis buffer containing 50 MM Tris, pH 7.4, 50 mM NaCl, 0.5% NP-40, 50 mM NaF, 1 mM Na₃VO₄, 1 mM phenylmethylsulfonyl fluoride, 25 μg/ml leupeptin, and 25 μg/ml aprotinin. The lysates were centrifuged at 15,004×g for 15 min and the supernatants were collected for protein concentration determination, SDS-PAGE, and western blot analysis with specific antibodies, as described.

Quantification of Apoptosis by ELISA

An ELISA-based kit (Roche Diagnostics Coup., Ind.) was used to quantitatively measure cytoplasmic histone-associated DNA fragments (mononucleosomes and oligonucleosomes), a measure of apoptosis, according to the manufacturer's instructions.

Xenograft model Nude mice (Harlan Sprague-Dawley, Indianapolis, Ind.) were maintained in accordance with the Institutional Animal Care and Use Committee (IACUC) procedures and guidelines. 10×10⁶ Raji (a human B-cell lymphoma line), or 5×10⁶ A2780 (a human ovarian cancer line) cells were suspended in 100 μL of phosphate buffered saline, mixed with 50% Matrigel, and implanted subcutaneously into the flanks of 5-week-old female nude mice. Clioquinol was dissolved in Intralipid® 20% (Fresenius Kabi Clayton, L. P., Clayton, N.C.), a soy-based lipid emulsion used to provide parenteral nutrition to human subjects. 1 mL of the dissolved clioquinol was given via i.p. injection 5 days a week, starting eight days after tumor inoculation. Control animals were given Intralipid, without Cloquinol. The tumor volume was assessed three times a week using calipers to measure the perpendicular diameters of the tumor, and calculated with the formula; v=l×(w)² (0.5), where v=tumor volume, l=length of tumor and w=width of donor (60). Animals' weights were also measured 3 times a week.

SOD1 Activity Analysis

SOD1 activity was assayed using the xanthine oxidase/cytochrome C method as previously described (61). Briefly, the control reaction was started by adding 1 U of xanthine oxidase (Sigma X 1875) to a reaction mixture containing 15 μM cytochrome c and 100 μM xanthine in 50 mM potassium phosphate, 0.1 mM EDTA, at pH 7.8, 25° C., in a 96-well plate. The plate was read every 15 seconds for up to 20 min at 550 nm. For SOD1 activity assay, one unit of purified bovine erythrocyte SOD 1 (Sigma, S-2515) or 15 μg of cellular extract was added to the reaction prior to the addition of xanthine oxidase. Cell extracts were prepared after 6 h treatment of Raji cells with 100 μM of clioquinol or 1 mM of diethyldithiocarbamate. Cells were pelleted, dissolved in PBS buffer, sonicated, and centrifuged to remove nuclei. Cytosolic protein concentrations were determined with Bio-Rad protein assay kit.

Cellular Zinc Level Measurements

A sensitive water-soluble salt form of a fluorescent zinc indicator was used for cellular zinc level measurement following the manufacture's protocol (FluoZin-3, Molecular Probes). A2780 (10,000/well) and Raji cells (30,000/well) were plated in a 96-well plate. 24 h after plating, the cells were treated with 30 μM clioquinol or 10 μM pyrithione for 1 h in the presence of increasing concentrations of zinc.

FluoZinc-3 (final concentration of 3 μM) was added and the cells were incubated for another 30 min at 37° C. After the incubation, cells were washed twice with fresh medium to remove any dye nonspecifically associated with the cell surface. Additional medium was added and the cells were incubated for a further 30 min before fluorescence measurements were commenced. The fluorescence was measured at 485nm/535nm (excitation/emission), using a Wallac 1420 Multilabel Counter (Perkin Elmer).

Statistics

IC₅₀ values were calculated with nonlinear regression analysis using Graphpad Prism 3.0 (Graphpad Software, Inc.).

### EXAMPLE 1

### Clioquinol Induces Cytotoxicity in Human Tumor Cell Lines

Inventors examined the effects of clioquinol on the viability of eight human cancer cell lines representing different tissues of origin. The cells studied included human B-cell lymphoma lines (DHL-4, Raji), breast (MCF-7, MDA-MB231), ovarian (A2780), cervical (SiHa), bladder (T24), and pancreatic (Mpanc-96) cancer cells. As shown in Table 1, treatment with clioquinol for 72 h reduced viability of all cell lines tested, with IC₅₀s ranging from 10-40 μM, concentrations that are similar to trough levels found in the blood of patients receiving oral clioquinol in a clinical trial for Alzheimer's disease (18). Time-course studies indicated that 8 h exposure to clioquinol was sufficient for full cytotoxicity towards A2780 cells to be demonstrated.

### EXAMPLE 2

### Tolerance of Clioquinol (CQ) by Humans

CQ was initially used widely around the world as an antibiotic until the late 1960s. At that time it was implicated in an outbreak of subacute myelo-optic neuropathy (SMON) in Japan (13, 20) and was taken off the market in Japan and the United States. The Japanese set up a multi-disciplinary commission to investigate SMON, and the commission concluded that clioquinol accounted for SMON in Japan (21). Others looking at the same data, however, did not agree with the commission's conclusions (22, 23, 24, 25).

Recently, Cherry et al. (14), Alzheimer's investigators, have shown that metal chelation could reduce amyloid deposits (26) and investigated whether chelation of metals such as zinc and copper might inhibit amyloid formation in a mouse model of Alzheimer's disease. They found that CQ could be administered safely to mice at doses of 30 mg/kg/day and that its administration resulted in a decrease in sedimentable AP amyloid in the brains of treated animals. They noted that CQ causes neuropathological changes that resemble those associated with vitamin B₁₂ deficiency (27) and that CQ has been demonstrated to cause a depletion of vitamin B₁₂ levels in neural tissue of mice (28). Administering vitamin B₁₂ may therefore be a way to prevent SMON. Second, by 1975 there were 10,000 cases of SMON reported in Japan but only 220 cases reported from the rest of the world (14), implying the existence of a co-factor (perhaps vitamin B₁₂ deficiency) that made Japanese more susceptible to SMON. A report from India, which did not ban clioquinol, noted no cases of SMON between 1977 and 1983 (29). Third, there has not been a clear demonstration of a dose-response relationship between CQ ingestion and the risk of developing SMON. Fourth, approximately 25% of 2465 Japanese patients with SMON had never taken CQ (30). Finally, CQ is currently available in some countries, including Canada and the United Kingdom.

A number of animal studies involving CQ have been performed over the past 34 years, and have demonstrated that high doses can induce neurotoxicity. Because CQ was recognized to bind certain metals, and vitamin B₁₂ contains cobalt, investigators studied the effects of CQ upon cobalt levels in mouse brains. Although cobalt levels were not altered by CQ, accumulation of vitamin B₁₂ in the brain was decreased by CQ administration and there was a decrease in S-adenosylmethionine levels, indicating a decrease in functional vitamin B₁₂ levels (28). The instant inventors found no reports in the literature concerning levels of vitamin B₁₂, folate, homocysteine, or methylmalonic acid in patients taking clioquinol or suffering from SMON.

Since the first report of benefit in a mouse model of Alzheimer's disease (14), CQ been shown to be of benefit in an animal model of Parkinson's disease (31). Its use in two human trials of Alzheimer's disease has been reported, without reported neurotoxicity. The first involved patients in Sweden, who took 10 or 80 mg of CQ a day for 21 days, without experiencing toxicity (17). The second (18) involved 36 patients in Australia who took CQ over a nine month period at progressively increasing doses (250, 500, and 750 mg a day), along with 100 μg of vitamin B₁₂ every 4 weeks. No neurotoxicity was found in the second clinical trial, which utilized doses significantly higher than the first.

### EXAMPLE 3

### Ionophoretic Properties of Clioquinol

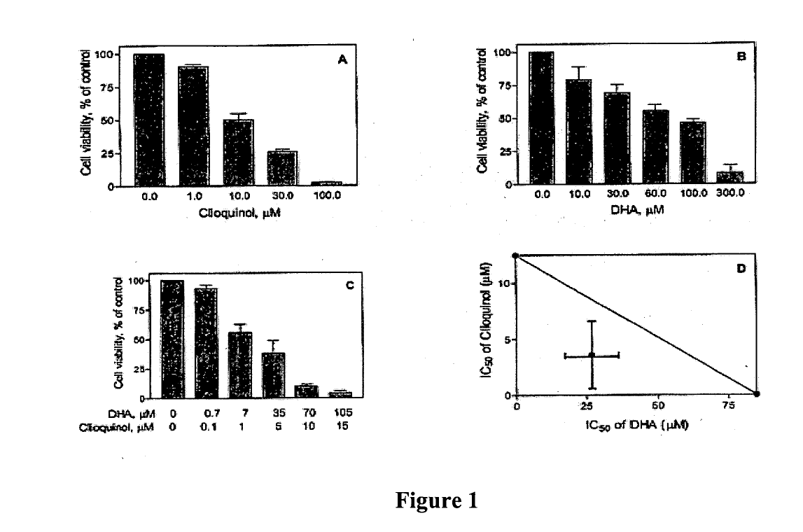

Bush et al. postulated that CQ chelates zinc and copper (32). The instant inventors felt that such a compound would inhibit SOD 1, which has been proposed as a target for anti-cancer drug therapy (33). Inventors studied CQ's effects on various human tumor cell lines when added alone or in addition to the n-3 polyunsaturated fatty acid, docosahexaenoic acid (DHA; 22:5, n-3), which is known to kill cells via oxidative mechanisms (34). As shown in Table 1 and FIG. 1, CQ inhibited growth of a variety of cancer cells lines, and exhibited synergism with DHA.

In FIG. 1 which shows clioquinol potentiated killing of tumor cells by DHA, Raji cells (a human B-cell Burkitt lymphoma line) were treated with clioquinol (A), DHA (B) or clioquinol plus DHA (C). Cell viability was determined with the MTS assay. IC₅₀ was calculated using nonlinear regression analysis (Prism 3.0). The concentration of clioquinol and DHA used in (C) was based on the ratio of IC₅₀s for clioquinol and DHA (12.4 μM and 85 μM, respectively). Shown in D is the isobolograrn analysis indicating a synergy of clioquinol and DHA in killing Raji cells. Data (n=3) are presented as means±S.E.M.

In vitro studies of the effect of CQ to purified bovine erythrocyte SOD 1 showed the inhibition of SOD 1 by CQ. Inventors however concluded that the cytotoxicity of CQ was not merely due to its inhibition of SOD1, because:
1) diethyldithiocarbamate, a recognized SOD 1 inhibitor (35) inhibited SOD I in treated cells to the same extent as CQ, but did not kill the cells;
2) CQ treatment of tumor cells does not lead to increased lipid peroxidation (as determined by the generation of thiobarbituric acid reactive substances), as would be expected if SOD 1 were significantly inhibited;
3) anti-oxidants such as vitamin E and Trolox, which block oxidant-induced cytoxicity caused by DHA, do not protect cells against CQ.

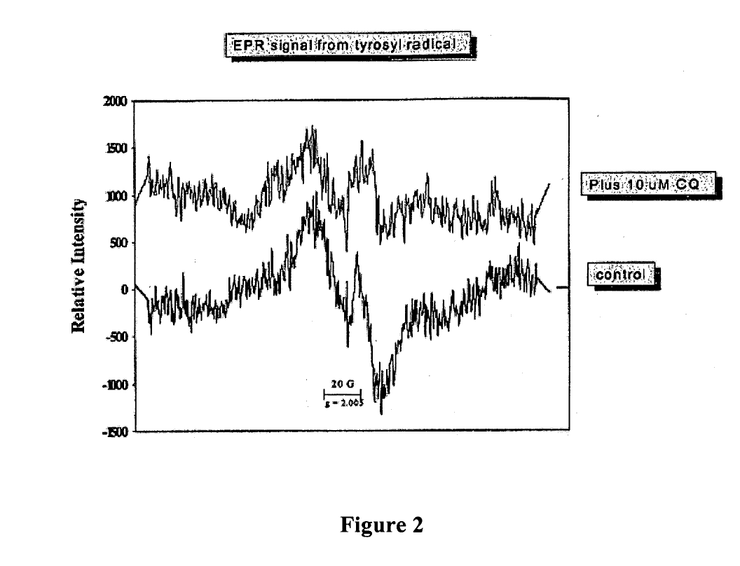

Inventors then considered whether CQ might be active because it binds intracellular iron, thereby inactivating the R2 subunit of the essential enzyme, ribonucleotide reductase (RR). Examination of CQ-treated cell extracts by electron spin resonance spectroscopy revealed in FIG. 2, loss of the active tyrosyl radical of RR, consistent with chelation of its iron. CQ was not able, however, to remove iron from recombinant R2. This suggests that clioquinol is inhibiting a step in the intracellular transport of iron to regenerate the tyrosyl radical in the R2 unit of ribonucleotide reductase, as has been reported for the siderophore desferri-exochelin 772SM (36).

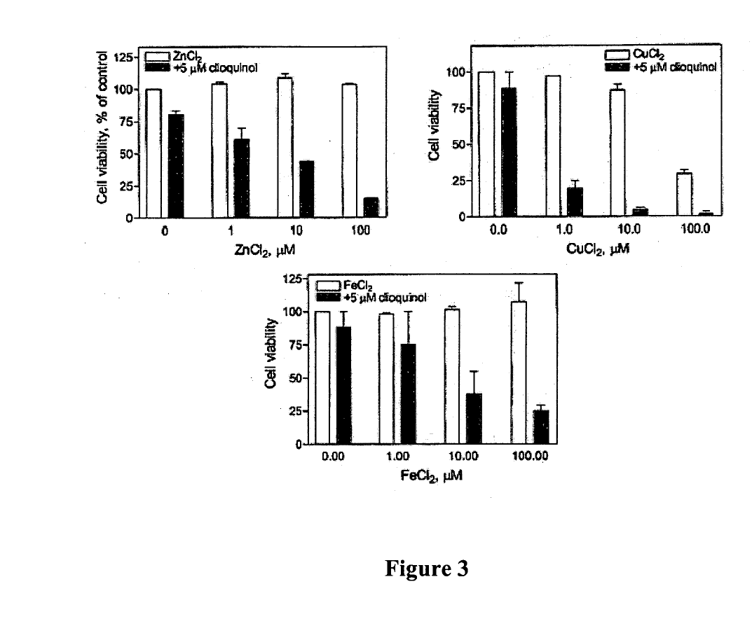

Inventors reasoned that the cytotoxic effects of a chelator would be overcome by an excess of the chelated metal. Inventors added increasing concentrations of copper, zinc or iron to cells treated with CQ. Cells were treated with ZnCl2, CuCl₂, and FeCl₂ along or premixed with 5 μM clioquinol for 72 h. Cell viability was expressed as a percentage of untreated cells. As shown in FIG. 3, each of these metals potentiated, rather than reversed CQ's cytotoxic effects, suggesting that CQ was not simply chelating a key metal.

Because copper and iron are known cytotoxic agents, the effect of added zinc was surprising. Zinc has not been considered to play a role in tumor cytotoxicity, and zinc deficiency appears to cause increased apoptosis, which can be blocked by zinc supplementation (37, 38). Because zinc is found in synaptic vesicles, neuroscientists have recently conducted more detailed studies of its cellular effects and have demonstrated the occurrence of zinc-mediated neurotoxicity under a variety of conditions (39, 40).

Given the surprising potentiation of zinc cytotoxicity by CQ, the inventors performed additional studies to determine if zinc might play a role in CQ-mediated cell killing. Inventors then studied the effects of the addition of zinc chelators. Since Ca⁺⁺ has a lower affinity for EDTA than Zn⁺⁺, the addition of ZNEDTA will not affect a zinc-dependent process, while ZNEDTA will inhibit it. Inventors found that the addition of CaEDTA (but not ZnEDTA) resulted in a decrease in CQ-mediated cell killing.

The added toxicity induced by higher zinc concentrations suggests that the zinc-CQ complex itself may not be the key toxic moiety, because a saturation effect would have been expected. An alternative explanation is that CQ is acting as a zinc ionophore, loading the cells with zinc, which in turn leads to cell death, perhaps by inducing mitochondrial toxicity (41). By using a fluorescent zinc indicator, inventors found that CQ is indeed an ionophore as shown in FIG. 3 in the ovarian cancer cell line, A2780. The zinc ionophore pyrithione was used as a positive control.

### EXAMPLE 4

### Pharmacokinetics and Blood Levels of CQ

CQ pharmacokinetics have been studied in animals and humans. In man, CQ is absorbed from the GI tract (with 25% excreted in the urine in 72 hours (19)), and from the skin (40% absorbed in 12 hours (42)). Its serum half-life is about 12 hours (19). Importantly, blood levels of CQ were measured in a recent Australian Alzheimer's trial (18) and doses of 750 mg a day result in trough, steady-state blood levels of 7.6 μg/mL (25 μM). Thus, oral dosing of CQ will give the prolonged blood levels equivalent to those required in vitro to inhibit tumor growth (Table 1).

### EXAMPLE 5

### Aptotic Properties of Clioquinol

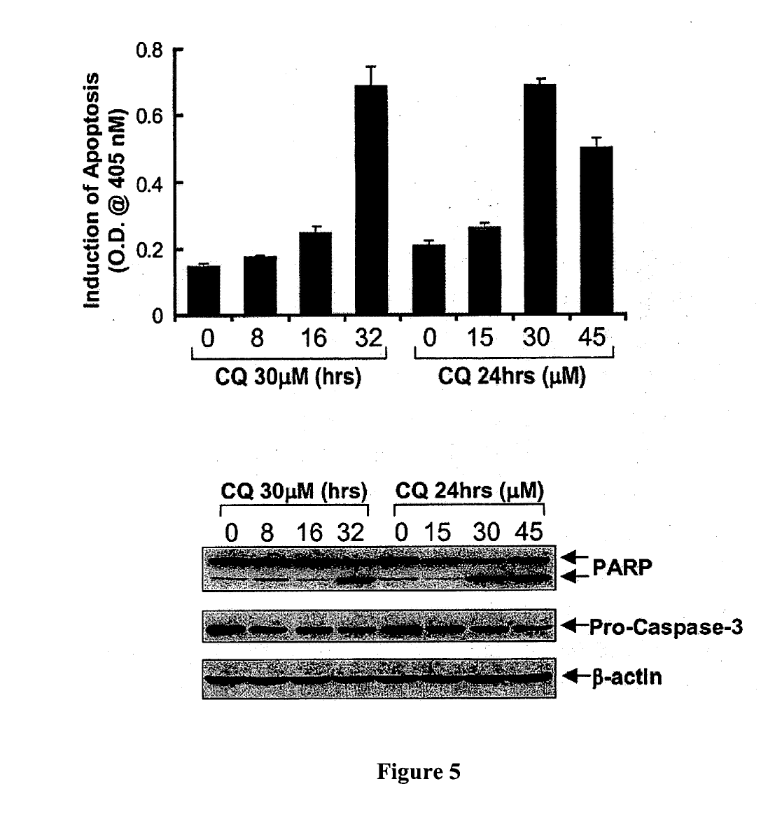

Raji cells were treated with clioquinol for 24 h and then lysed. Cellular extracts were analyzed using an ELISA-based assay (Roche Diagnostics Corp., Indianapolis, Ind.) that quantifies release into the cytoplasm of histone-associated DNA fragments (mononucleosomes and oligonucleosomes), a measure of apoptosis. In addition, the lysates were subjected to SDS-PAGE followed by Western blotting using an anti-poly (ADP-ribose) polymerase (PARP) monoclonal antibody (clone C-2-10) to detect PARP cleavage, a sign of caspase-dependent apoptosis. These studies demonstrated, as shown in FIG. 5, that clioquinol induces apoptosis of Raji cells through caspase-dependent mechanisms.

In FIG. 5, showing clioquinol induced apoptosis in Raji cells through caspase dependent mechanism, panel A shows induction of apoptosis by clioquinol. Raji cells were treated with 30 μM clioquinol for indicated times or 24 h with clioquinol at the indicated concentrations. Cell lysates were prepared and apoptosis determined using an ELISA that quantified cytoplasmic DNA-histone complexes. In panel B, an equal amount of protein for each sample as prepared in (A) was resolved by 8% SDS electrophoresis, followed by Western blot analysis with specific antibodies directed against PARP, caspase-3, or P-actin.

### EXAMPLE 6

### Clioquinol Inhibits Tumor Growth in Nude Mice

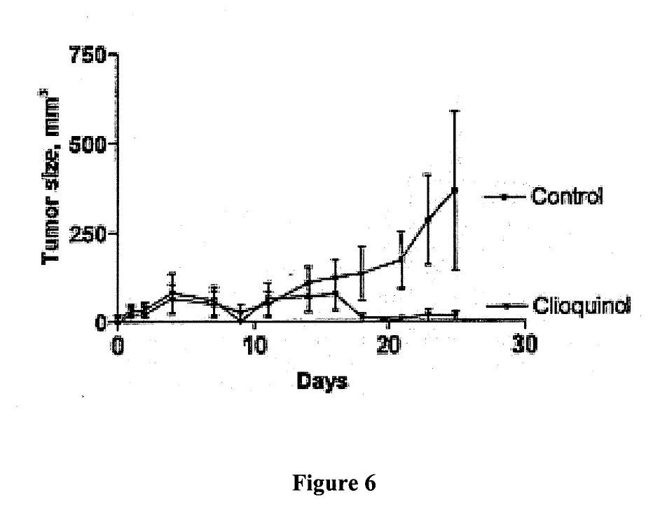

Nude mice were injected subcutaneously with Raji cells. The control group received ip injections of 20% Intralipid, an FDA-approved soy-based emulsion given intravenously to humans, and the treatment group received the same emulsion containing 28 mg/kg clioquinol (a dose chosen on the basis of the work of Cherny (14), and the preferred dosing regimen of the present invention, although it is contemplated that clioquinol, alone or in combination with other active ingredients will be used in an effective amount to inhibit tumor growth. No animals exhibited signs of toxicity. As shown in FIG. 6, administration of clioquinol resulted in inhibition of tumor growth.

In FIG. 6 showing the effects of clioquinol on growth of a human E-cell lymphoma line in nude mice, Raji cells (10×10⁶/100 μl) were injected subcutaneously into the flanks of nude mice. Clioquinol (28 mg/kg) was given 5 days a week, starting one week after implantation, Tumor size was calculated and expressed as mm . (Control group, n=4; clioquinol group, n=5).

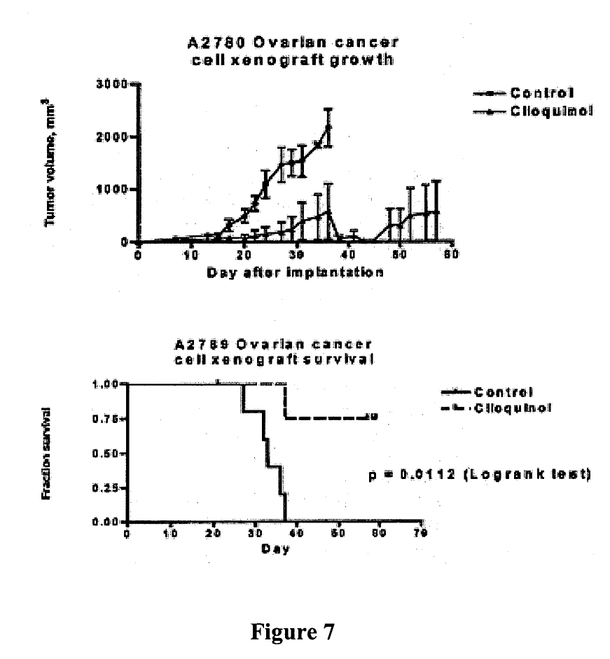

In a separate experiment, nude mice were injected with the ovarian cancer cell line, A2780 and treated similarly. Mice were killed when the tumor reached 10% of their body weight. As shown in FIG. 7, clioquinol inhibited tumor growth and prolonged survival of treated mice.

In FIG. 7 showing the effects of clioquinol on growth of a human ovarian cancer cell line in nude mice, A2780 were injected subcutaneously into the flanks of nude mice. Clioquinol (28 mg/kg) was given 5 days a week, starting one week after implantation. Tumor size was calculated and expressed as mm . (Control group, n=5; clioquinol group, n=5).

Although not a newly synthesized compound, clioquinol serves as a prototype for a novel class of anti-cancer agents, the divalent cation ionophores and this invention is not limited to the use of clioquinol but relates to the use of ionophores, in general, as a novel class of anti-tumor agents. Preferred ionophores of the present invention include but are not limited to clioquinol, chloroxine (5, 7-dichloro -8-hydroxyquinolone), oxine (8-hydroxyquinolone), pyrithione (2-mercaptopyridine 1-oxide). While metal chelating compounds have been used for anti-cancer purposes, ionophores are a distinct subgroup because they can give up their bound metal. Tetrathiomolybdate is a high-affinity copper-chelating compound which has been shown to aid in excretion of copper in animals (43) and humans (44) with Wilson's disease, and is being taken through preclinical and clinical trials (45). Clioquinol, on the other hand, has a modest affinity for copper (log K₁=8.9) and zinc (log K₁=7) (46), and a pK₁ that is close to that of the cytosol (7.9) (47), which apparently combine favorably and allows it to transport ions intracellularly.

It is contemplated that these ionophores can be used alone or in combination with metals. Clioquinol can be used with metals as adjuncts. Copper and zinc are two preferred metals. Mixing clioquinol with metals before their joint administration is one option. An alternative is to use albumin as a carrier. There are several reasons to consider albumin in this context. Clioquinol binds albumin with high affinity (10⁻⁸ M) (48); zinc and copper bind albumin with high affinity (Zn(II), 10⁻⁷ M; CU(II), 10⁻¹¹ M) (49); albumin is relatively excluded from the brain by the blood-brain-barrier; and albumin is preferentially taken up by tumors.

Regarding the preferential uptake of albumin by tumors, a small literature going back to the mid-twentieth century indicates that tumors selectively take up albumin (reviewed in (50)). The best study (51) utilized albumin that was radiolabeled in such a way that the radioactive moiety could not be reutilized (52) and found tumor uptake of albumin was very significant—equal to, or greater than (depending upon the model studied) that of the liver. Using albumin to target tumors is an approach being explored by many groups for imaging (53, 54) and drug delivery (55, 56).

Albumin is found within cancer cells in clinical specimens. Although the literature indicates that tumor cells take up albumin, much of the work was based upon radioactive tracer studies performed before non-degradable tracers were developed. Inventors therefore sought to establish that human cancers contain intracellular albumin. Using a commercially available monoclonal antibody prepared to human albumin, the present inventors have found albumin in 12 of 12 breast and colon cancers.

The present inventors therefore contemplate a method of treating cancer using ionophores such as clioquinol as a single agent; ionophores such as clioquinol administered simultaneously with zinc, copper, iron or manganese; ionophores such as clioquinol administered with albumin-metal complexes, and said ionophores may be administered at various times prior to or preferably after administering the metal or the albumin-metal complexes. Each of these treatment modalities may also be used in combination with polyunsaturated fatty acids anti-cancer agents for synergistic chemotherapeutic effects.

### EXAMPLE 7

### Anti-Angiogenic Properties of Clioquinol, Pyrithione, and Pyrrolidone Dithiocarbamate

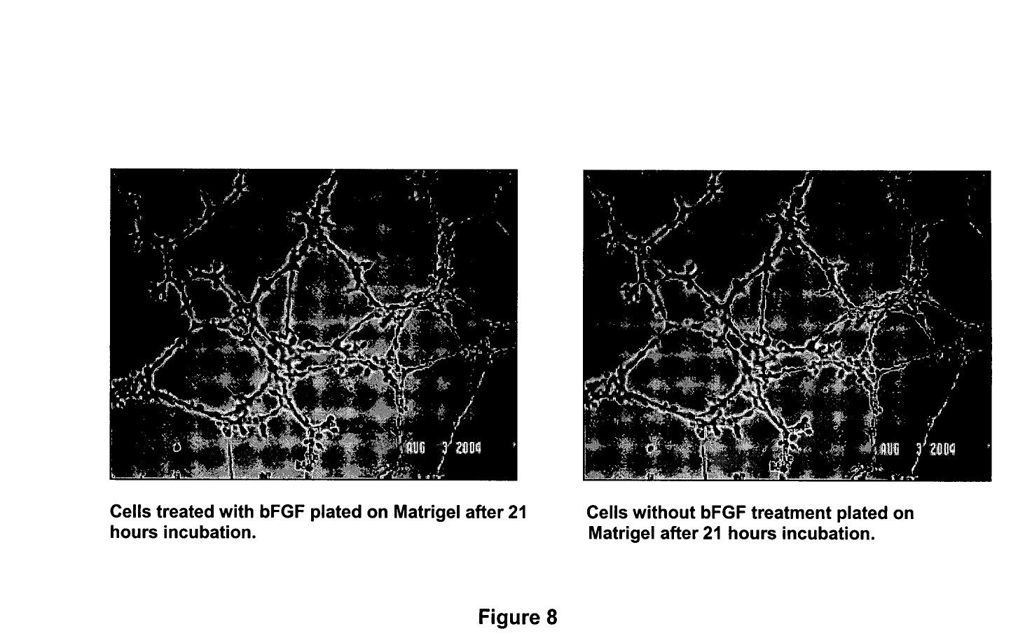

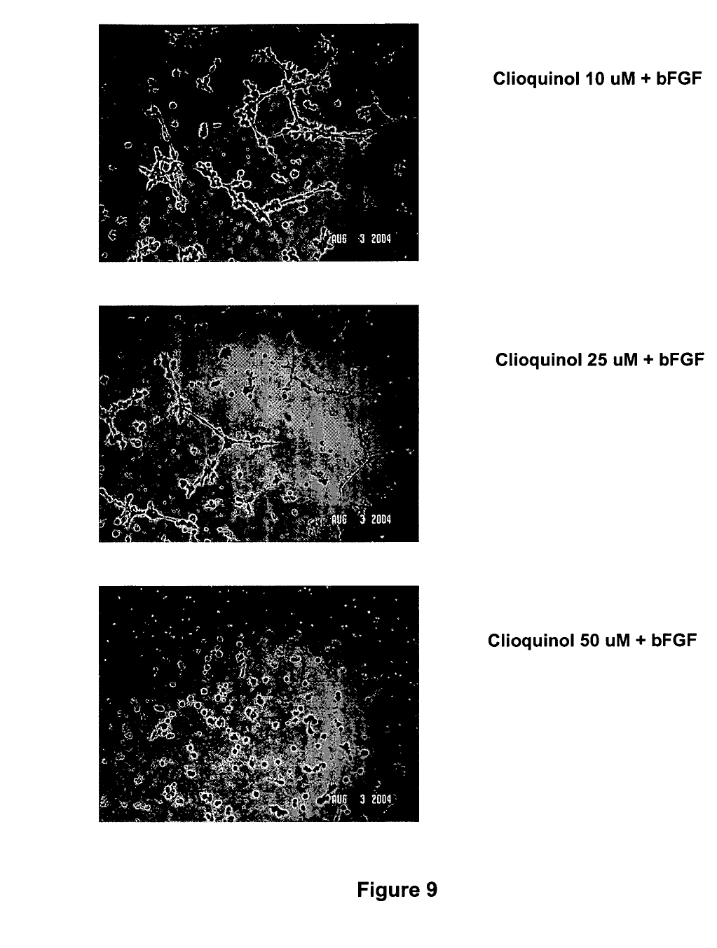

The anti-angiogenic effect of the compounds of the present invention have been verified using the chorio-allantoic membrane model. Also, said anti-angiogenic properties have also been verified using inhibition of endothelial cell tube formation assay using standard techniques known in the art. An immortalized endothelial cell line (EAhy.926) was plated on Matrigel at a density of 15,000 per well in the absence or presence of the drugs indicated overnight at 37° C. Branch formation was used as a measure of tube formation. Each value was determined by averaging the number of branches per field (250×) in 3 representative fields. The final bFGF concentration was 5 ng/mL. FIG. 8 shows a comparative study of endothelial cell tube formation between cells treated with bFGF and without bFGF plated on Matrigel after 21 hours of incubation. FIG. 8 clearly shows the stimulative effect of bFGF (basic Fibroblast growth factor) on endothelial cell tube formation. As shown in FIG. 9, endothelial cell tube formation is inhibited in a dose dependent manner by clioquinol.

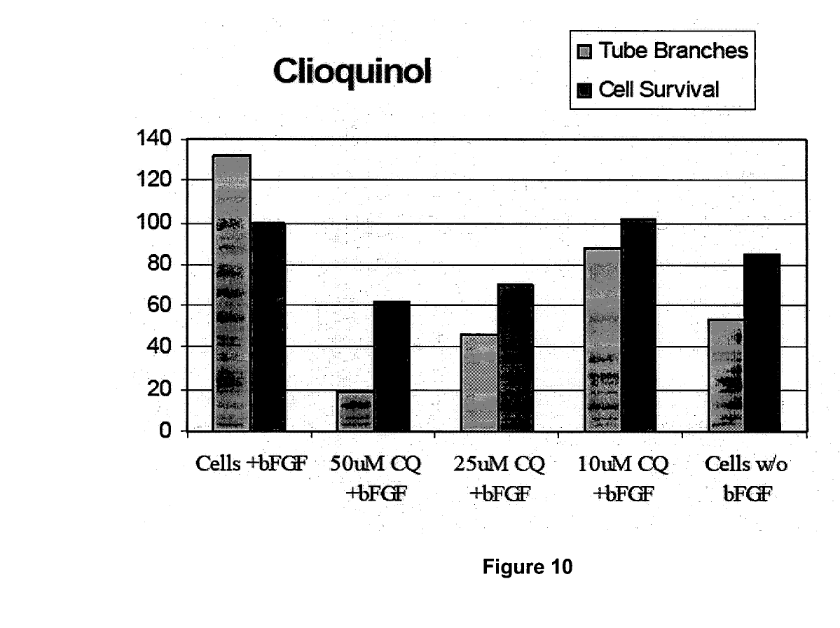

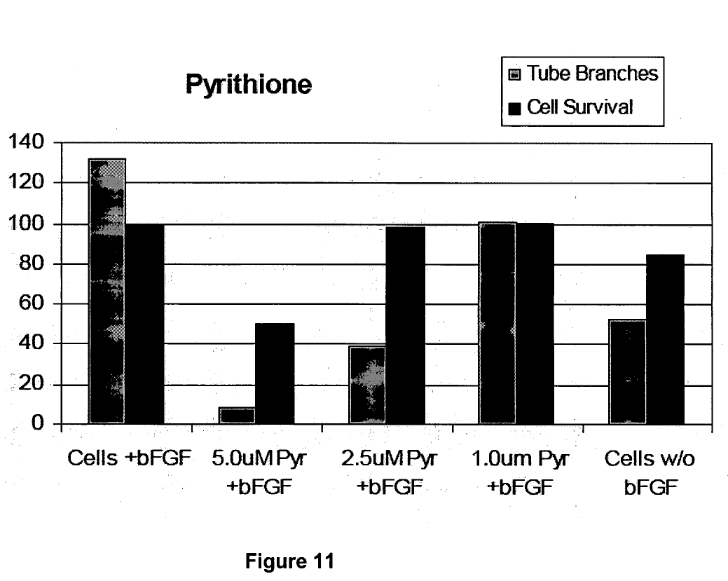

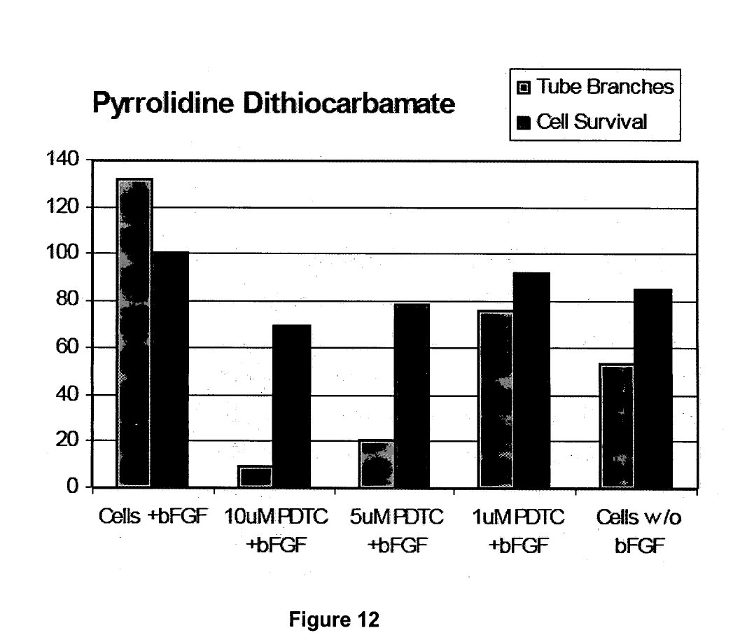

FIG. 10 shows a correlation between cell survival and tube branches and again shows the anti-angiogenic effect of clioquinol. Similarly, FIG. 11 and 12 show a correlation between cell survival and tube branches and the anti-angiogenic effect of pyrithione and pyrrolidone dithiocarbamate.

Thus present inventors have found that ionophores such as clioquinol and related compounds have anti-angiogenic effects and do so, among other subcellular and molecular activities, by disrupting endothelial cell tube formation. Thus, angiogenic dependent disease states represent attractive targets for therapeutic intervention for certain disease states.

In particular, the anti-angiogenic effect of clioquinol and other compounds of the present invention represents an attractive chemotherapeutic target. Angiogenesis, is the mechanism by which new capillaries are formed from existing vessels. When required, the vascular system has the potential to generate new capillary networks in order to maintain the proper functioning of tissues and organs. In the adult, however, angiogenesis is fairly limited, occurring only in the process of wound healing and neovascularization of the endometrium during menstruation. See Merenmies, J., Parada, L. F., Henkemeyer, M., Cell Growth & Differentiation, 8, 3-10 (1997). On the other hand, unwanted angiogenesis is a hallmark of several diseases, such as retinopathies, psoriasis, rheumatoid arthritis, age-related macular degeneneration, and cancer (solid tumors). Folkman, Nature Med., 1, 27-31 (1995).

Thus, it is also provided in accordance with the invention, a pharmaceutical composition containing ionophores such as clioquinol and other compounds of the present invention and their derivatives or a pharmaceutically acceptable salts thereof; or prodrug, or pharmaceutically active metabolites thereof, or a pharmaceutically acceptable salts of the metabolites or prodrugs, and the therapeutic use of the composition in treating diseases mediated by angiogenesis, such as cancer, as well as other disease states associated with unwanted angiogenesis and/or cellular proliferation, such as diabetic retinopathy, neovascular glaucoma, rheumatoid arthritis, and psoriasis.

As aptototic agents and anti-angiogenic agents, it is an object of the present invention to provide a method of treating proliferative disorders in mammals, especially humans, marked by unwanted proliferation of endogenous tissue. Compounds of the present invention may be used for treating subjects having a disorder associated with excessive cell proliferation, e.g., cancers, psoriasis ( see for e.g. Naldi et al. Br J Dermatol. 2005. April, 152(4); 597-615), immunological disorders involving undesired proliferation of leukocytes, and restenosis and other smooth-muscle disorders. Furthermore, such compounds may be used to prevent de-differentiation of post-mitotic tissue and/or cells.

Diseases or disorders associated with uncontrolled or abnormal cellular proliferation which are susceptible to treatment by compound of the present invention include, but are not limited to, the following:
a variety of cancers, including, but not limited to, carcinoma, hematopoietic tumors of lymphoid lineage, hematopoietic tumors of myeloid lineage, tumors of mesenchymal origin, tumors of the central and peripheral nervous system and other tumors including melanoma, seminoma and Kaposi's sarcoma and the like.
a disease process which features abnormal cellular proliferation, e.g., benign prostatic hyperplasia, familial adenomatosis polyposis, neuro-fibromatosis, atherosclerosis, pulmonary fibrosis, arthritis, psoriasis, glomerulonephritis, restenosis following angioplasty or vascular surgery, hypertrophic scar formation, inflammatory bowel disease, transplantation rejection, endotoxic shock, and fungal infections.
defective apoptosis-associated conditions, such as cancers (including but not limited to those types mentioned hereinabove), viral infections (including but not limited to herpesvirus, poxvirus, Epstein-Barr virus, Sindbis virus and adenovirus), prevention of AIDS development in HIV-infected individuals, autoimmune diseases (including but not limited to systemic lupus erythematosus, rheumatoid arthritis, psoriasis, autoimmune mediated glomerulonephritis, inflammatory bowel disease and autoimmune diabetes mellitus), neurodegenerative disorders (including but not limited to Alzheimer's disease, amyotrophic lateral sclerosis, retinitis pigmentosa, Parkinson's disease, AIDS-related dementia, spinal muscular atrophy and cerebellar degeneration), myelodysplastic syndromes, aplastic anemia, ischemic injury associated with myocardial infarctions, stroke and reperfusion injury, arrhythmia, atherosclerosis, toxin-induced or alcohol related liver diseases, hematological diseases (including but not limited to chronic anemia and aplastic anemia), degenerative diseases of the musculoskeletal system (including but not limited to osteroporosis and arthritis), aspirin-sensitive rhinosinusitis, cystic fibrosis, multiple sclerosis, kidney diseases and cancer pain.

Therapeutically effective amounts of the agents of the invention may be used to treat diseases mediated by angiogenesis or hyperproliferation of cells. An “effective amount” is intended to mean that amount of an agent that, when administered to a mammal in need of such treatment, is sufficient to effect treatment for a disease mediated by the activity of one or more kinases. Thus, e.g., a therapeutically effective amount of ionophores, salt, active metabolite or prodrug thereof is a quantity sufficient to inhibit the hyper proliferation of cancer cells or angiogenesis such that a disease condition which is mediated by that activity is reduced or alleviated. “Treating” is intended to mean at least the mitigation of a disease condition in a mammal, such as a human, that is affected, at least in part, angiogenesis or cellular hyperproliferation, and includes: preventing the disease condition from occurring in a mammal, particularly when the mammal is found to be predisposed to having the disease condition but has not yet been diagnosed as having it; modulating and/or inhibiting the disease condition; and/or alleviating the disease condition.

The amount of the present compounds administered to the subject will depend on the type and severity of the disease or condition and on the characteristics of the subject, such as general health, age, sex, body weight and tolerance to drugs. The skilled artisan will be able to determine appropriate dosages depending on these and other factors. Effective dosages for commonly used anti-cancer drugs and radiation therapy are well known to the skilled person.

Techniques for formulation and administration of the compounds of the instant invention can be found in Remington: the Science and Practice of Pharmacy, 19^{th} edition, Mack Publishing Co., Easton, Pa. (1995). The compositions of the invention may be manufactured in manners generally known for preparing pharmaceutical compositions, e.g., using conventional techniques such as mixing, dissolving, granulating, dragee-making, levigating, emulsifying, encapsulating, entrapping or lyophilizing. Pharmaceutical compositions may be formulated in a conventional manner using one or more physiologically acceptable carriers, which may be selected from excipients and auxiliaries that facilitate processing of the active compounds into preparations which can be used pharmaceutically.

Proper formulation is dependent upon the route of administration chosen. For injection, the agents of the invention may be formulated into aqueous solutions, preferably in physiologically compatible buffers such as Hanks's solution, Ringer's solution, or physiological saline buffer. For transmucosal administration, penetrants appropriate to the barrier to be permeated are used in the formulation. Such penetrants are generally known in the art.

For oral administration, the compounds can be formulated readily by combining the active compounds with pharmaceutically acceptable carriers known in the art. Such carriers enable the compounds of the invention to be formulated as tablets, pills, dragees, capsules, liquids, gels, syrups, slurries, suspensions and the like, for oral ingestion by a patient to be treated. Pharmaceutical preparations for oral use can be obtained using a solid excipient in admixture with the active ingredient (agent), optionally grinding the resulting mixture, and processing the mixture of granules after adding suitable auxiliaries, if desired, to obtain tablets or dragee cores. Suitable excipients include: fillers such as sugars, including lactose, sucrose, mannitol, or sorbitol; and cellulose preparations, for example, maize starch, wheat starch, rice starch, potato starch, gelatin, gum, methyl cellulose, hydroxypropylmethyl-cellulose, sodium carboxymethylcellulose, or polyvinylpyrrolidone (PVP). If desired, disintegrating agents may be added, such as crosslinked polyvinyl pyrrolidone, agar, or alginic acid or a salt thereof such as sodium alginate.

Dragee cores are provided with suitable coatings. For this purpose, concentrated sugar solutions may be used, which may optionally contain gum arabic, polyvinyl pyrrolidone, Carbopol gel, polyethylene glycol, and/or titanium dioxide, lacquer solutions, and suitable organic solvents or solvent mixtures. Dyestuffs or pigments may be added to the tablets or dragee coatings for identification or to characterize different combinations of active agents.

Pharmaceutical preparations which can be used orally include push-fit capsules made of gelatin, as well as soft, sealed capsules made of gelatin and a plasticizer, such as glycerol or sorbitol. The push-fit capsules can contain the active ingredients in admixture with fillers such as lactose, binders such as starches, and/or lubricants such as talc or magnesium stearate, and, optionally, stabilizers. In soft capsules, the active agents may be dissolved or suspended in suitable liquids, such as fatty oils, liquid paraffin, or liquid polyethylene glycols. In addition, stabilizers may be added. All formulations for oral administration should be in dosages suitable for such administration. For buccal administration, the compositions may take the form of tablets or lozenges formulated in conventional manner.

For administration intranasally or by inhalation, the compounds for use according to the present invention are conveniently delivered in the form of an aerosol spray presentation from pressurized packs or a nebuliser, with the use of a suitable propellant, e.g., dichlorodifluoromethane, trichlorofluoromethane, dichlorotetrafluoroethane, carbon dioxide or other suitable gas. In the case of a pressurized aerosol the dosage unit may be determined by providing a valve to deliver a metered amount. Capsules and cartridges of gelatin for use in an inhaler or insufflator and the like may be formulated containing a powder mix of the compound and a suitable powder base such as lactose or starch.

The compounds may be formulated for parenteral administration by injection, e.g., by bolus injection or continuous infusion. Formulations for injection may be presented in unit-dosage form, e.g., in ampoules or in multi-dose containers, with an added preservative. The compositions may take such forms as suspensions, solutions or emulsions in oily or aqueous vehicles, and may contain formulatory agents such as suspending, stabilizing and/or dispersing agents.

Pharmaceutical formulations for parenteral administration include aqueous solutions of the active compounds in water-soluble form. Additionally, suspensions of the active agents may be prepared as appropriate oily injection suspensions. Suitable lipophilic solvents or vehicles include fatty oils such as sesame oil, or synthetic fatty acid esters, such as ethyl oleate or triglycerides, or liposomes. Aqueous injection suspensions may contain substances which increase the viscosity of the suspension, such as sodium carboxymethyl cellulose, sorbitol, or dextran. Optionally, the suspension may also contain suitable stabilizers or agents which increase the solubility of the compounds to allow for the preparation of highly concentrated solutions.

For administration to the eye, the active agent is delivered in a pharmaceutically acceptable ophthalmic vehicle such that the compound is maintained in contact with the ocular surface for a sufficient time period to allow the compound to penetrate the corneal and internal regions of the eye, including, for example, the anterior chamber, posterior chamber, vitreous body, aqueous humor, vitreous humor, cornea, iris/ciliary, lens, choroid/retina and sclera. The pharmaceutically acceptable ophthalmic vehicle may be an ointment, vegetable oil, or an encapsulating material. A compound of the invention may also be injected directly into the vitreous and aqueous humor.

Alternatively, the active ingredient may be in powder form for constitution with a suitable vehicle, e.g., sterile pyrogen-free water, before use. The compounds may also be formulated in rectal compositions such as suppositories or retention enemas, e.g., containing conventional suppository bases such as cocoa butter or other glycerides.

The compounds may also be formulated as a depot preparation. Such long-acting formulations may be administered by implantation (for example, subcutaneously or intramuscularly) or by intramuscular injection. Thus, for example, the compounds may be formulated with suitable polymeric or hydrophobic materials (for example, as an emulsion in an acceptable oil) or ion-exchange resins, or as sparingly soluble derivatives, for example, as a sparingly soluble salt.

A pharmaceutical carrier for hydrophobic compounds is a cosolvent system comprising benzyl alcohol, a nonpolar surfactant, a water-miscible organic polymer, and an aqueous phase. The cosolvent system may be a VPD co-solvent system. VPD is a solution of 3% w/v benzyl alcohol, 8% w/v of the nonpolar surfactant polysorbate 80, and 65% w/v polyethylene glycol 300, made up to volume in absolute ethanol. The VPD co-solvent system (VPD:5W) contains VPD diluted 1:1 with a 5% dextrose in water solution. This co-solvent system dissolves hydrophobic compounds well, and itself produces low toxicity upon systemic administration. Naturally, the proportions of a co-solvent system may be varied considerably without destroying its solubility and toxicity characteristics. Furthermore, the identity of the co-solvent components may be varied: for example, other low-toxicity nonpolar surfactants may be used instead of polysorbate 80; the fraction size of polyethylene glycol may be varied; other biocompatible polymers may replace polyethylene glycol, e.g. polyvinyl pyrrolidone; and other sugars or polysaccharides may be substituted for dextrose.

Alternatively, other delivery systems for hydrophobic pharmaceutical compounds may be employed. Liposomes and emulsions are known examples of delivery vehicles or carriers for hydrophobic drugs. Certain organic solvents such as dimethylsulfoxide also may be employed, although usually at the cost of greater toxicity. Additionally, the compounds may be delivered using a sustained-release system, such as semipermeable matrices of solid hydrophobic polymers containing the therapeutic agent. Various sustained-release materials have been established and are known by those skilled in the art. Sustained-release capsules may, depending on their chemical nature, release the compounds for a few weeks up to over 100 days. Depending on the chemical nature and the biological stability of the therapeutic reagent, additional strategies for protein stabilization may be employed.

The pharmaceutical compositions also may comprise suitable solid- or gel-phase carriers or excipients. Examples of such carriers or excipients include calcium carbonate, calcium phosphate, sugars, starches, cellulose derivatives, gelatin, and polymers such as polyethylene glycols.

Some of the compounds of the invention may be provided as salts with pharmaceutically compatible counter ions. Pharmaceutically compatible salts may be formed with many acids, including hydrochloric, sulfuric, acetic, lactic, tartaric, malic, succinic, etc. Salts tend to be more soluble in aqueous or other protonic solvents than are the corresponding free-base forms.

Those skilled in the art will understand that the preferred embodiments described above are illustrative and explanatory and may be subject to modification without departing from the scope and spirit of the invention. The inventors, accordingly, hereby state their intentions to rely upon the Doctrine of Equivalents in order to protect the full scope of their invention. Further, all the references cited in this specification are incorporated herein by reference.

### REFERENCES

1. Desoize B. Metals and metal compounds in carcinogenesis. In Vivo 2003;17(6):529-39.
2. Toyokuni S. Iron-induced carcinogenesis: the role of redox regulation. Free Radic Biol Med 1996;20(4):553-66.
3. Theophanides T, Anastassopoulou J. Copper and carcinogenesis. Crit Rev Oncol Hematol 2002;42(1):57-64.
4. Brewer G J. Copper control as an antiangiogenic anticancer therapy: lessons from treating Wilson's disease. Exp Biol Med (Maywood) 2001;226(7):665-73.
5. Lovejoy D B, Richardson D R. Iron chelators as anti-neoplastic agents; current developments and promise of the PIH class of chelators. Curr Med Chem 2003;10(12): 1035-49.
6. Pan Q, Kleer C G, van Golen K L, et al, Copper deficiency induced by tetrathiomolybdate suppresses tumor growth and angiogenesis. Cancer Res 2002;62(17):4854-9.
7. Brewer G J, Dick R D, Grover D K, et al. Treatment of metastatic cancer with tetrathiomolybdate, an anticopper, antiangiogenic agent: Phase I study. Clin Cancer Res 2000;6(l):1-10.
8. Redman B G, Esper P, Pan Q, et al. Phase II trial of tetrathiomolybdate in patients with advanced kidney cancer. Clin Cancer Res 2003;9(5): 1666-72.
9. Abeysinghe R D, Greene B T, Haynes R, et al. p53-independent apoptosis mediated by tachpyridine, an anti-cancer iron chelator. Carcinogenesis 2001;22(10):1607-14.
10. Fan L, Iyer J, Zhu S, et al. Inhibition of N-myc expression and induction of apoptosis by iron chelation in human neuroblastoma cells. Cancer Res 2001 ;61(3): 1073-9.
11. Pan Q, Bao L W, Merajver S D. Tetrathiomolybdate inhibits angiogenesis and metastasis through suppression of the NFkappaB signaling cascade. Mol Cancer Res 2003;1(10):7016.
12. Hollingshead R. The Dihalogen and Trihalogen Derivatives. Oxine and Its Derivatives 1956;III:740.
13. Tsubaki T, Honma Y, Hoshi M. Neurological syndrome associated with clioquinol. Lancet 1971;1(7701):696-7.
14. Chemy R A, Atwood C S, Xilinas M E, et al. Treatment with a copper-zinc chelator markedly and rapidly inhibits beta-arnyloid accumulation in Alzheimer's disease transgenic mice. Neuron 2001;30(3):665-76.
15. Begin M E, Ells G, Horrobin D F. Polyunsaturated fatty acid-induced cytotoxicity against tumor cells and its relationship to lipid peroxidation. J Natl Cancer Inst 1988;80(3):188-94.
16. Callaway T R, Edrington T S, Rychlik J L, et al. Ionophores: their use as ruminant growth promotants and impact on food safety. Curr Issues Intest Microbiol 2003;4(2):43-51.
17. Regland B, Lehmann W, Abedini I, et al. Treatment of Alzheimer's disease with clioquinol. Dement Geriatr Cogn Disord 2001;12(6):408-14.
18. Ritchie C W, Bush Al, Mackinnon A, et al. Metal-protein attenuation with iodochlorhydroxyquin (clioquinol) targeting Abeta amyloid deposition and toxicity in Alzheimer disease: a pilot phase 2 clinical trial. Arch Neurol 2003;60(12):1685-91.
19. Jack D B, Riess W. Pharmacokinetics of iodochlorhydroxyquin in man. J Pharm Sci 1973;62(12): 1929-32.
20. Kono R. Subacute myelo-optico-neuropathy, a new neurological disease prevailing in Japan. Jpn J Med Sci Biol 1971;24(4):195-216.
21. Kono R. Introductory review of subacute myelo-optico-neuropathy (SMON) and its studies done by the SMON Research Commission. Jpn J Med Sci Biol 1975;28 Suppl: 1-21.
22. Meade T W. Subacute myelo-optic neuropathy and clioquinol. An epidemiological case-history for diagnosis. Br J Prev Soc Med 1975;29(3):157-69.
23. Clifford Rose F, Gawel M, Clioquinol neurotoxicity: an overview. Acta Neurol Scand Suppl 1984;100:137-45.
24. Shigematsu 1. Sub acute myelo-optico-neuropathy (SMON) and clioquinol: -A response to doubts about clioquinol causation theory. Jpn J Med Sci Biol 1975;28 Suppl :35-55.
25. Tabira T. Clioquinol's return: cautions from Japan. Science 2001;292(5525):2251-2.
26. Cherny R A, Legg ST, McLean C A, et al. Aqueous dissolution of Alzheimer's disease Abeta amyloid deposits by biometal depletion. J Biol Chem 1999;274(33):23223-8.
27. Shiraki H. The neuropathology of subacute myelo-optico-neuropathy, “SMON”, in the humans: —with special reference to the quinoform intoxication. Jpn J Med Sci Biol 1975;28 Suppl:101-64.
28. Yassin M S, Ekblom J, Xilinas M, Gottfries C G, Oreland L. Changes in uptake of vitamin B(12) and trace metals in brains of mice treated with clioquinol. J Neurol Sci 2000; 173(l):40-4.
29. Wadia N H. SMON as seen from Bombay. Acta Neurol Scand Suppl 1984;100:159-64.
30. Nakae K, Yamamoto S, Shigematsu 1, Kono R. Relation between subacute myelo-optic neuropathy (S.M.O.N.) and clioquinol: nationwide survey. Lancet 1973;1(7796):171-3.
31. Kaur D, Yantiri F, Rajagopalan S, et al. Genetic or pharmacological iron chelation prevents MPTP-induced neurotoxicity in vivo: a novel therapy for Parkinson's disease. Neuron 2003;37(6):899-909.
32. Bush Al. The metallobiology of Alzheimer's disease. Trends Neurosci 2003;26(4):207-14.
33. Huang P, Feng L, Oldham E A, Keating M J, Plunkett W. Superoxide dismutase as a target for the selective killing of cancer cells. Nature 2000;407(6802):390-5.
34. Begin M E, Ells G, Das U N, Horrobin D F. Differential killing of human carcinoma cells supplemented with n-3 and n-6 polyunsaturated fatty acids. J Nat) Cancer Inst 1986;77(5): 1053-62.
35. Heikkila R E, Cabbat F S, Cohen G. Inactivation of superoxide dismutase by several thiocarbamic acid derivatives. Experientia 1978;34(12):1553-4.
36. Hodges Y K, Antholine W E, Horwitz L D. Effect on ribonucleotide reductase of novel lipophilic iron chelators: the desferri-exochelins. Biochem Biophys Res Commun 2004;315(3):595-8.
37. Zalewski PD, Forbes I J, Betts W H. Correlation of apoptosis with change in intracellular labile Zn(II) using zinquin [(2-methyl-8-p-toluenesulphonamido-6-quinolyloxy)acetic acid], a new specific fluorescent probe for Zn(II). Biochem J 1993;296 ( Pt 2):403-8.
38. Sunderman F W, Jr. The influence of zinc on apoptosis. Ann Clin Lab Sci 1995;25(2):13442.
39. Choi D W, Koh J Y. Zinc and brain injury. Annu Rev Neurosci 1998;21 :347-75.
40. Lobner D, Canzoniero L M, Manzerra P, et al. Zinc-induced neuronal death in cortical neurons. Cell Mol Biol (Noisy-le-grand) 2000;46(4):797-806.
41. Dineley K E, Votyakova T V, Reynolds U. Zinc inhibition of cellular energy production: implications for mitochondria and neurodegeneration. J Neurochern 2003;85(3):563-70.
42. Stohs S J, Ezzedeen F W, Anderson A K, Baldwin J N, Makoid M C. Percutaneous absorption of iodochlorhydroxyquin in humans. J Invest Dermatol 1984;82(2):195-8.
43. Suzuki K T. Disordered copper metabolism in LEC rats, an animal model of Wilson disease: roles of metallothionein. Res Commun Mol Pathol Pharmacol 1995;89(2):221-40.
44. Brewer G J, Hedera P, Kluin K J, et al. Treatment of Wilson disease with ammonium tetrathiomolybdate: III. Initial therapy in a total of 55 neurologically affected patients and follow-up with zinc therapy, Arch Neurol 2003;60(3):379-85.
45. Goodman V L, Brewer G J, Merajver S D. Copper deficiency as an anti-cancer strategy. Endocr Relat Cancer 2004;1 1(2):255-63.
46. Padmanabhan G, Becue I, Smith J B. Clioquinol. Analytical Profiles of Drug Substances 1989;18:57-90.
47. 6.0 NSRDV. NIST Critically selected stability constants of metal complexes. In.
48. Tanaka H, Tamura Z. Analysis of chinoform binding to human serum albumin by an improved partition equilibrium method. J Pharmacobiodyn 1986;9(12):1015-22.
49. Masuoka J, Hegenauer J, Van Dyke B R, Saltman P. Intrinsic stoichiometric equilibrium constants for the binding of zinc(II) and copper(II) to the high affinity site of serum albumin. J Biol Chem 1993;268(29):21533-7.
50. Stehle G, Sinn H, Wunder A, et al. Plasma protein (albumin) catabolism by the tumor itself—implications for tumor metabolism and the genesis of cachexia. Grit Rev Oncol Hematol 1997;26(2):77-100.
51. Wunder A, Stehle G, Sinn H, et al. Enhanced albumin uptake by rat tumors. Int J Oncol 1997;1 1:497-507.
52. Strobel J L, Cady S G, Borg T K, Terracio L, Baynes J W, Thorpe S R. Identification of fibroblasts as a major site of albumin catabolism in peripheral tissues. J Biol Chem 1986;261(17):7989-94.
53. McDonald M A, Watkin I L L, Small particulate gadolinium oxide and gadolinium oxide albumin microspheres as multimodal contrast and therapeutic agents. Invest Radiol 2003;38(6):305-10.
54. Kiessling F, Fink C, Hansen M, et al. Magnetic resonance imaging of nude mice with heterotransplanted high-grade squamous cell carcinomas: use of a low-loaded, covalently bound Gd-Hsa conjugate as contrast agent with high tumor affinity. Invest Radiol 2002;37(4): 193-8.
55. Dennis M S, Zhang M, Meng Y G, et al. Albumin binding as a general strategy for improving the pharmacokinetics of proteins. J Biol Chem 2002;277(38):35035-43.
56. Chuang V T, Kragh-Hansen U, Otagiri M. Pharmaceutical strategies utilizing recombinant human serum albumin. Pharm Res 2002;19(5):569-77.
57. Liu, B. and Fan, Z. The monoclonal antibody 225 activates caspase-8 and induces apoptosis through a tumor necrosis factor receptor family-independent pathway. Oncogene, 20: 3726-3734, 2001.
58. Liu, B., Fang, M., Schmidt, M., Lu, Y., Mendelsohn, J., and Fan, Z. Induction of apoptosis and activation of the caspase cascade by anti-EGF receptor monoclonal antibodies in DiFi human colon cancer cells do not involve the c-jun N-terminal kinase activity. Br J Cancer, 82: 1991-1999, 2000.
59. Liu, B., Peng, D., Lu, Y., Jin, W., and Fan, Z. A novel single amino acid deletion caspase-8 mutant in cancer cells that lost proapoptotic activity. J Biol Chem, 277: 30159-30164, 2002.
60. Sun, J., Blaskovich, M. A., Knowles, D., Qian, Y., Ohkanda, J., Bailey, R. D., Hamilton, A. D., and Sebti, S. M. Antitumor efficacy of a novel class of non-thiol-containing peptidomimetic inhibitors of farnesyltransferase and geranylgeranyltransferase I: combination therapy with the cytotoxic agents cisplatin, Taxol, and gemcitabine. Cancer Res, 59: 4919-4926, 1999.
61. Okado-Matsumoto, A. and Fridovich, 1. Assay of superoxide dismutase: cautions relevant to the use of cytochrome c, a sulfonated tetrazolium, and cyanide. Anal Biochem, 298: 337-342, 2001.

1. A method of treating cancer comprising administering to a mammal in need of such treatment, an effective amount of ionophore or pharmaceutically acceptable salts thereof.

2. The method of claim 1, wherein the ionophore is at least one selected from the group consisting of clioquinol, chloroxine (5, 7-dichloro -8-hydroxyquinolone), oxine (8-hydroxyquinolone), and pyrithione (2-mercaptopyridine 1-oxide).

3. The method of claim 1, wherein the ionophore is at least one selected from the group consisting of carbamodithioic acid, prolinedithiocarbamate, disulfiram, diethyldithiocarbamate, pyrrolidine dithiocarbamate, dimethyldithiocarbamate, diethylthiocarbamate, diethyldithiocarbamate ion, dimethylthiocarbamate, and dibenzyldithiocarbamate.

4. The method of claim 1, wherein the ionophore is clioquinol.

5. A method of treating cancer comprising administering to a mammal in need of such treatment, a pharmaceutical composition comprising an effective amount of ionophore or pharmaceutically acceptable salts thereof and an effective amount of transition metals.

6. The method of claim 5, wherein the ionophore is at least one selected from the group consisting of clioquinol, chloroxine (5, 7-dichloro -8-hydroxyquinolone), oxine (8-hydroxyquinolone), and pyrithione (2-mercaptopyridine 1-oxide).

7. The method of claim 5, wherein the ionophore is at least one selected from the group consisting of carbamodithioic acid, prolinedithiocarbamate, disulfiram, diethyldithiocarbamate, pyrrolidine dithiocarbamate, dimethyldithiocarbamate, diethylthiocarbamate, diethyldithiocarbamate ion, dimethylthiocarbamate, and dibenzyldithiocarbamate.

8. The method of claim 5, wherein the ionophore is clioquinol.

9. The method of any one of claims 5-8, wherein the transition metal is at least one selected from the group consisting of zinc, copper, manganese, and iron.

10. A method of treating cancer comprising administering to a mammal in need of such treatment, a pharmaceutical composition comprising an effective amount of ionophore or pharmaceutically acceptable salts thereof, an effective amount of transition metals, and an effective amount of albumin.

11. The method of claim 10, wherein the ionophore is at least one selected from the group consisting of clioquinol, chloroxine (5, 7-dichloro -8-hydroxyquinolone), oxine (8-hydroxyquinolone), and pyrithione (2-mercaptopyridine 1-oxide).

12. The method of claim 10, wherein the ionophore is at least one selected from the group consisting of carbamodithioic acid, prolinedithiocarbamate, disulfiram, diethyldithiocarbamate, pyrrolidine dithiocarbamate, dimethyldithiocarbamate, diethylthiocarbamate, diethyldithiocarbamate ion, dimethylthiocarbamate, and dibenzyldithiocarbamate.

13. The method of claim 10, wherein the ionophore is clioquinol.

14. The method of any one of claims 10-13, wherein the transition metal is at least one selected from the group consisting of zinc, copper, manganese, and iron.

15. The method of claim 10, wherein the albumin is in the form of a metal-albumin complex or an ionophore-albumin complex.

16. A method of treating cancer comprising administering to a mammal in need of such treatment, a pharmaceutical composition comprising an effective amount of ionophore or pharmaceutically acceptable salts thereof, and an effective amount of polyunsaturated fatty acids.

17. The method of claim 16, wherein the pharmaceutical composition further comprises one or more transition metals.

18. The method of claim 17, wherein the pharmaceutical composition further comprises albumin.

19. The method of any one of claims 16-18, wherein the polyunsaturated fatty acid is at least one selected from the group consisting of docosahexaenoic acid, conjugated docosahexaenoic acid, eicosapentaenoic acid, conjugated eicosapentaenoic acid, alpha or beta—eleostearic acid, punicic acid, parinaric acid, and linoleic acid isomers.

20. A pharmaceutical composition for the treatment of cancer comprising an effective amount of ionophore or pharmaceutically acceptable salts thereof and a suitable pharmaceutical carrier.

21. The composition of claim 20, wherein the ionophore is at least one selected from the group consisting of clioquinol, chloroxine (5, 7-dichloro -8-hydroxyquinolone), oxine (8-hydroxyquinolone), and pyrithione (2-mercaptopyridine 1-oxide).

22. The composition of claim 20, wherein the ionophore is at least one selected from the group consisting of carbamodithioic acid, prolinedithiocarbamate, disulfiram, diethyldithiocarbamate, pyrrolidine dithiocarbamate, dimethyldithiocarbamate, diethylthiocarbamate, diethyldithiocarbamate ion, dimethylthiocarbamate, and dibenzyldithiocarbamate.

23. The composition of claim 20, wherein the ionophore is clioquinol.

24. A pharmaceutical composition for the treatment of cancer comprising an effective amount of ionophore or pharmaceutically acceptable salts thereof and an effective amount of transition metals and pharmaceutically acceptable carrier.

25. The pharmaceutical composition of claim 24, wherein the ionophore is at least one selected from the group consisting of clioquinol, chloroxine (5, 7-dichloro -8-hydroxyquinolone), oxine (8-hydroxyquinolone), and pyrithione (2-mercaptopyridine 1-oxide).

26. The pharmaceutical composition of claim 24, wherein the ionophore is at least one selected from the group consisting of carbamodithioic acid, prolinedithiocarbamate, disulfiram, diethyldithiocarbamate, pyrrolidine dithiocarbamate, dimethyldithiocarbamate, diethylthiocarbamate, diethyldithiocarbamate ion, dimethylthiocarbamate, and dibenzyldithiocarbamate.

27. The pharmaceutical composition of claim 24, wherein the ionophore is clioquinol.

28. The pharmaceutical composition of claims 24-27, wherein the transition metal is at least one selected from the group consisting of zinc, copper, manganese, and iron.

29. A pharmaceutical composition for the treatment of cancer comprising an effective amount of ionophore or pharmaceutically acceptable salts thereof, an effective amount of transition metals, and an effective amount of albumin and pharmaceutically acceptable carrier.

30. The pharmaceutical composition of claim 29, wherein the ionophore is at least one selected from the group consisting of clioquinol, chloroxine (5, 7-dichloro -8-hydroxyquinolone), oxine (8-hydroxyquinolone), and pyrithione (2-mercaptopyridine 1-oxide).

31. The pharmaceutical composition of claim 29, wherein the ionophore is at least one selected from the group consisting of carbamodithioic acid, prolinedithiocarbamate, disulfiram, diethyldithiocarbamate, pyrrolidine dithiocarbamate, dimethyldithiocarbamate, diethylthiocarbamate, diethyldithiocarbamate ion, dimethylthiocarbamate, and dibenzyldithiocarbamate.

32. The pharmaceutical composition of claim 29, wherein the ionophore is clioquinol.

33. The pharmaceutical composition any one of claims 29-32, wherein the transition metal is at least one selected from the group consisting of zinc, copper, manganese, and iron.

34. The pharmaceutical composition of claim 29, wherein the albumin is in the form of a metal-albumin complex or an ionophore-albumin complex.

35. A pharmaceutical composition for the treatment of cancer comprising an effective amount of ionophore or pharmaceutically acceptable salts thereof, and an effective amount of polyunsaturated fatty acids and a pharmaceutically acceptable carrier.

36. The pharmaceutical composition of claim 35, wherein the pharmaceutical composition further comprises one or more transition metals.

37. The pharmaceutical composition of claim 35, wherein the pharmaceutical composition further comprises albumin.

38. The pharmaceutical composition of any one of claims 35-37, wherein the polyunsaturated fatty acid is at least one selected from the group consisting of docosahexaenoic acid, conjugated docosahexaenoic acid, eicosapentaenoic acid, conjugated eicosapentaenoic acid, alpha or beta—eleostearic acid, punicic acid, parinaric acid, and linoleic acid isomers.

39. A pharmaceutical composition for treating a disease state associated with uncontrolled cellular proliferation comprising:
i. an ionophore or a pharmaceutically acceptable salt, prodrug or pharmaceutically active metabolite or a pharmaceutically acceptable salt of a metabolite or prodrug thereof, and
ii. a pharmaceutically acceptable carrier.

40. The pharmaceutical composition of claim 39, wherein the ionophore is at least one selected from the group consisting of clioquinol, chloroxine (5, 7-dichloro -8-hydroxyquinolone), oxine (8-hydroxyquinolone), and pyrithione (2-mercaptopyridine 1-oxide).

41. The pharmaceutical composition of claim 39, wherein the ionophore is at least one selected from the group consisting of carbamodithioic acid, prolinedithiocarbamate, disulfiram, diethyldithiocarbamate, pyrrolidine dithiocarbamate, dimethyldithiocarbamate, diethylthiocarbamate, diethyldithiocarbamate ion, dimethylthiocarbamate, and dibenzyldithiocarbamate.

42. The pharmaceutical composition of claim 39, wherein the ionophore is clioquinol.

43. A method of treating a disease state or disorder associated with uncontrolled cellular proliferation comprising administering to a subject in need thereof a therapeutically effective amount of an ionophore, or a pharmaceutically acceptable salt of a compound thereof; or a prodrug or pharmaceutically active metabolite of a compound thereof or a pharmaceutically acceptable salt of a prodrug thereof.

44. The method of claim 43, wherein the ionophore is at least one selected from the group consisting of clioquinol, chloroxine (5, 7-dichloro -8-hydroxyquinolone), oxine (8-hydroxyquinolone), and pyrithione (2-mercaptopyridine 1-oxide).

45. The method of claim 43, wherein the ionophore is at least one selected from the group consisting of carbamodithioic acid, prolinedithiocarbamate, disulfiram, diethyldithiocarbamate, pyrrolidine dithiocarbamate, dimethyldithiocarbamate, diethylthiocarbamate, diethyldithiocarbamate ion, dimethylthiocarbamate, and dibenzyldithiocarbamate.

46. The method of claim 43, wherein the ionophore is clioquinol.
